(19) United States
(12) Patent Application Publication
(10) Pub. No.: US 2006/0058218 A1
(43) Pub. Date: Mar. 16, 2006

(54) Solid phase conjugation of complexing agents and targeting moieties

(75) Inventors: Faisal A. Syud, Clifton Park, NY (US); John B. Brogan, Niskayuna, NY (US); Daniel Joshua Kramer, Ballston Lake, NY (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: General Electric Company, Schenectady, NY (US)

(21) Appl. No.: 10/937,323

(22) Filed: Sep. 10, 2004

(51) Int. Cl. A61K 38/16; C07K 1/02

(52) U.S. Cl. 514/2; 530/333

(57) ABSTRACT

There is provided a technique for conjugating one or more complexing agents with a targeting moiety, such as natural amino acids, unnatural amino acids, peptides, peptide nucleic acids, nucleotides, and analogs and derivatives thereof. The one or more complexing agents are conjugated at one or more free amino groups of the targeting moiety while the moiety is attached to a solid substrate.

### FIELD OF THE INVENTION

Embodiments of the invention relate generally to the synthesis of radiolabeled diagnostic and therapeutic pharmaceuticals, and to the compounds made from the synthesis. More particularly, embodiments of the invention relate to the controlled solid phase conjugation of targeting moieties such as amino acids, peptides, peptide nucleic acids, nucleotides, and analogs and derivatives thereof with complexing agents such as tetraazacyclododecane and tetraazacyclotetradecane chelates.

### BACKGROUND OF THE INVENTION

Radiopharmaceutical compounds are increasingly used in diagnostic and therapeutic medical procedures. Radiopharmaceuticals are pharmaceutically acceptable compounds that carry at least one radioactive, signal-generating element that is typically bound to a biomolecular carrier, for example a targeting moiety. The radioactive, signal-generating element may produce a signal detectable by radiological diagnostic equipment. For example, positron emission tomography (PET) is an imaging technique that detects radiation emitted from radioactive tracers, or imaging contrast agents, injected into the body. Additionally, because the radiation emitted by the radioactive element may have a toxic effect on tissues, the radiopharmaceutical may be utilized to achieve beneficial therapeutic effects. For example, a radiopharmaceutical may be used as a chemotherapy drug to kill cancerous tissues.

In either case, it may be desirable to direct the radiopharmaceuticals to specific structures in the body or sites of physiological functions. When used as an imaging contrast, localization of the radiopharmaceutical at a specific structure or site in the body helps to produce more highly contrasted, and therefore more easily readable and accurate, images. When used as a therapeutic agent, localization of the radiopharmaceutical at a specific structure or site in the body concentrates the deleterious effects of the radiopharmaceutical in the structures or sites that are to be treated and helps prevent unwanted harmful effects at other structures and sites in the body.

Radioactive metallic ions such as ⁶⁴Cu are convenient sources of radiation for radiopharmaceuticals. In order to bind radioactive metallic ions in radiopharmaceuticals, compounds capable of complexing with a metal, “complexing agents,” such as cyclic chelating compounds, may be conjugated to the biomolecular carrier. 1,4,7,10-tetraazacyclododecane-1,4,7,10-tetraacetic acid (DOTA) and 1,4,8,11-tetraazacyclotetradecane-1,4,8,11-tetraacetic acid (TETA) are exemplary macrocyclic tetraaza chelating compounds that may be used to bind radioactive metallic ions in diagnostic and therapeutic radiopharmaceuticals. The process of binding the radioactive metallic ion with the complexing agent of the radiopharmaceutical is called “radiolabeling.” Various methods exist to radiolabel the radiopharmaceutical. In general, radiolabeling may be performed either before the complexing agent is conjugated to the biomolecular carrier (“prelabeling”) or after the complexing agent is conjugated to the biomolecular carrier.

For example, U.S. Pat. No. 4,707,352, the disclosure of which is incorporated herein in its entirety, discloses a method of radiolabeling comprising contacting an unlabeled therapeutic or diagnostic agent with an ion transfer material having the radioactive metal ion bound thereto. The ion transfer material has a weaker binding affinity for the radioactive metal ion than does a chelating portion of the unlabeled agent. Prior to contacting, the chelating portion is either unchelated or is chelated with a second metal ion having a binding affinity with the chelating portion less than the binding affinity of the radioactive metal ion.

Another exemplary radiolabeling method is disclosed in U.S. Pat. No. 5,958,374, the disclosure of which is incorporated herein in its entirety, which describes a prelabeling process for ⁹⁰Yttrium and ¹¹¹Indium comprising (a) reacting a chelating agent that has a trivalent chelating group and at least one pendant linker group that is capable of covalently binding to a ligand, with ⁹⁰Yttrium or ¹¹¹Indium to form an electrically neutral ⁹⁰Yttrium or ¹¹¹ Indium chelate; (b) purifying the chelate from the reaction mixture of (a); and (c) reacting the purified chelate of (b) with the ligand to form the complex. Polyazamacrocyclic moieties are identified as exemplary chelating groups capable of complexing with radionuclides.

If desired, radiopharmaceuticals may be stabilized in order to avoid radiolytic self-decomposition of the compound, which reduces the shelf life of the radiopharmaceutical and may cause unwanted side reactions in experiments performed with the radiopharmaceutical. Some approaches to minimizing radiolytic self-decomposition are reducing the molar activity of the compound, dispersing the compound in a solvent or solid diluent, adding free-radical inhibitors, adding inhibitors against chemical decomposition, and storing the compound at low temperatures.

U.S. Pat. No. 4,793,987, the disclosure of which is incorporated herein in its entirety, discloses exemplary stabilizers for radioactively labeled organic compounds. The stabilizers are derived from pyridine and inhibit radiolytic self-decomposition of radiolabeled amino acids, nucleotides, thionucleotides, nucleosides, steroids, lipids, fatty acids, peptides, carbohydrates, proteins, and nucleic acids.

U.S. Pat. No. 5,843,396, the disclosure of which is incorporated herein in its entirety, discloses stabilizing compounds selected from the group consisting of certain heteroaryls, substituted aryls, and alkylamines.

Targeting moieties often are employed as the bimolecular carrier in the radiopharmaceutical in order to direct the radiopharmaceutical to specific structures in the body or sites of physiological functions. A targeting moiety is a compound with structure or site specific reactivity. Exemplary targeting moieties include antibodies or antibody fragments, oligopeptides, polypeptides, receptor-binding molecules, DNA fragments, RNA fragments, and analogs and derivatives thereof.

Peptide nucleic acid (PNA) is another exemplary targeting moiety that may be used in a radiopharmaceutical. U.S. Pat. No. 6,395,474, the disclosure of which is incorporated herein by reference in its entirety, describes PNA as an analogue of DNA in which the phosphodiester backbone of DNA is replaced with a pseudo-peptide such as N-(2-amino-ethyl)-glycine. Methylenecarbonyl linkers attach DNA, RNA, or synthetic nucleobases to the polyamide backbone. PNA, obeying Watson-Crick hydrogen bonding rules, mimics the behavior of DNA and RNA by binding to complementary nucleic acid sequences such as those found in DNA, RNA, and other PNAs. An exemplary radiopharmaceutical utilizing PNA may bind, for example, to a specific mutated nucleic acid sequence found in the DNA of a cancerous tumor. An exemplary PET image produced using the PNA-based contrast agent may thereby show the location of the tumor having that specific genetic mutation. An exemplary therapeutic PNA-based radiopharmaceutical may direct lethal radiation to cancerous tissues.

Peptide nucleic acids, oligopeptides, and polypeptides are commonly synthesized using solid phase peptide synthesis (SPPS) techniques. In general, SPPS involves attaching a first amino acid to a solid phase substrate such as a polymeric resin. The alpha carbonyl group of an additional amino acid is coupled to the terminal amino group of the first amino acid via a condensation reaction. The terminal amino group of the additional amino acid and side chains of both the first and additional amino acid are protected during coupling to prevent unwanted reactions. Subsequent to coupling, the terminal amino group of the additional amino acid itself may be deprotected and coupled with a alpha carbonyl group of another additional amino acid. The process of deprotecting the amino acid attached to the polymer substrate and coupling with an additional amino acid may be repeated many times in order to add more amino acids to the peptide chain. When the desired peptide chain is produced, the peptide chain is deprotected and cleaved from the substrate.

In the case of a PNA, specially designed amino acids that form the pseudo-peptide backbone of PNA are coupled during SPPS. U.S. Pat. No. 6,713,602, the disclosure of which is incorporated herein by reference in its entirety, discloses peptide nucleic acids generally comprising ligands such as naturally occurring DNA bases attached to a peptide backbone. An especially preferred monomer for the synthesis of PNAs is the amino acid of the formula (I): 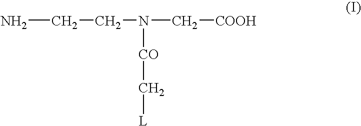
where L is selected from the nucleobases thymine, adenine, cytosine, guanine, and uracil.

Oligonucleotides such as DNA, RNA, and analogs and derivatives thereof also may be synthesized using solid phase techniques. DNA, for example, is synthesized by attaching a first nucleotide base to a solid phase substrate. The 5′-hydroxyl group of the phosphodiester backbone of the DNA nucleotide is protected during attachment to the substrate. The protecting group is removed and an activated additional nucleotide base is conjugated to the first nucleotide base via a condensation reaction between the 5′-hydroxyl group of the first nucleotide and the phosphorus linkage of the additional nucleotide to form a weak phosphite linkage. Unreacted first nucleotide base is capped by acetylation to exclude it from further synthetic elaboration. The weak phosphite linkage then is converted to a stronger phosphate linkage. The process of deprotecting the 5′-hydroxyl group of the nucleotide attached to the polymer substrate and coupling with an additional nucleotide may be repeated many times in order to add more nucleotide bases to the DNA. When the desired DNA sequence is produced, the DNA is deprotected and cleaved from the substrate.

Complexing agents such as DOTA and TETA may be bound to a targeting species by reaction with a free carboxylic group of the complexing agent. However, some complexing agents have an excess of carboxylic groups. DOTA and TETA, for example, each have four free carboxylic groups open for conjugation with a free amino group. This may result in oversubstitution of the targeting species.

One method to accomplish single-substitution reaction of DOTA or TETA with a targeting species, for example, is by reacting in solution an excess of DOTA or TETA with the targeting species. However, this method still produces a mixture of di-, tri-, and tetra-conjugated DOTAs and TETAs which then must be separated from the mono-conjugated product through high precision liquid chromatography (HPLC) or similar separation technologies. HPLC and other similar methods are expensive, slow, and difficult, thereby limiting their utility in mass production processes. Furthermore, this method results in the loss of expensive targeting species that are unintentionally incorporated into di-, tri-, and tetra-conjugated DOTAs and TETAs.

The description herein of problems and disadvantages of known apparatus, methods, and compositions is not intended to limit the invention to the exclusion of these known entities. Indeed, embodiments of the invention may include one or more of the known apparatus, methods, and compositions without suffering from the disadvantages and problems noted herein.

### SUMMARY OF THE INVENTION

There is a need for a solid phase synthetic method to selectively conjugate complexing agents with targeting moieties.

In accordance with a feature of an embodiment, there is provided a method for the conjugation of one or more complexing agents with a targeting moiety. The targeting moiety comprises at least one monomeric unit and may be attached to a solid phase substrate to form a targeting moiety-substrate component. The complexing agent may be conjugated to the targeting moiety-substrate component at one or more free amino groups of the targeting moiety—substrate component to form a complexing targeting moiety-substrate component.

Still further features and advantages of embodiments of the present invention are identified in the ensuing description.

### DETAILED DESCRIPTION OF THE INVENTION

The following description is intended to convey a thorough understanding of embodiments of the present invention by providing a number of specific embodiments and details involving solid phase conjugation of targeting moieties and complexing agents. It is understood, however, that the various embodiments of the present invention are not limited to these specific embodiments and details, which are exemplary only. It is further understood that one possessing ordinary skill in the art, in light of known systems and methods, would appreciate the use of the invention for its intended purposes and benefits in any number of alternative embodiments.

One embodiment provides a method for the conjugation of one or more complexing agents and a targeting moiety. The targeting moiety may comprise at least one monomeric unit and may be attached to a solid phase substrate to form a targeting moiety—substrate component. The complexing agent may be conjugated to the targeting moiety—substrate component at one or more free amino groups of the targeting moiety—substrate component to form a complexing targeting moiety—substrate component.

The targeting moieties used in the present invention may be any applicable monomeric or polymeric biological entity with structure or site specific reactivity in the body. Applicable targeting moieties include, but are not limited to, natural amino acids, unnatural amino acids, peptides, peptide nucleic acids, nucleotides, and analogs and derivatives thereof. It may be preferable that at least one of the monomeric units of the targeting moiety be a lysine, lysine derivative, or lysine analog.

An exemplary amino acid targeting moiety is illustrated in (1) of Synthesis I. The amino acid has a terminal amino group, a carboxyl group, and a side chain denoted as R. 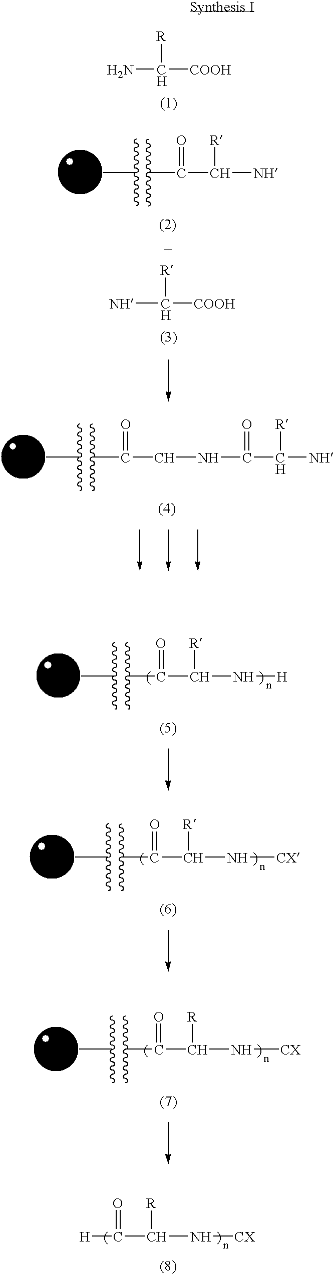
In Synthesis I above, R is independently selected from hydrogen and side groups covalently bonded to α-carbons of an α-amino acids (it is believed that there are twenty known naturally occurring α-amino acids); R′ is a protected form of R, CX is a complexing agent, CX is a protected form of CX, NH is a protected amino group, and n is an integer, preferably in the range of from about 4 to about 20, inclusive. Although Synthesis I illustrates an amino acid similar to one of the twenty known naturally occurring α-amino acids, one skilled in the art will understand that other α-amino acids may likewise be utilized in place of the illustrated first amino acid (1) and additional amino acids (3), in accordance with the principles of the present invention, as described herein. A preferred synthetic amino acid that may be used is the N-(2-amino-ethyl)-glycine backbone of PNAs. Additionally, analogs and derivatives of natural and synthetic amino acids, peptides, peptide nucleic acids, and nucleotides all may be used as targeting moieties in accordance with the present invention. One skilled in the art will appreciate other applicable targeting moieties that may be utilized, in accordance with the guidelines herein.

The substrate may be any applicable solid phase substrate, in accordance with the limitations herein. Substrates used for the solid phase synthesis of polypeptides, for example, are preferred substrates. Such substrates are often polymeric, resin-based substrates. One such preferred polymeric substrate is a beaded matrix of slightly cross-linked styrene-divinylbenzene copolymer, the cross-linked copolymer having been formed by the pearl polymerization of styrene monomer to which has been added a mixture of divinylbenzenes. A level of 1-2% cross-linking is most preferred. Another preferred polymer substrate is (methyl-benzhydryl) amine polystyrene resin, which is often used during the solid phase synthesis of PNAs. A more preferred substrate that also commonly is used for the solid phase synthesis of PNAs is 5-(4-Fmoc-aminoethyl-3,5-dimethoxyphenoxy)-valeric acid-MBHA resin (commercially available from Applied Biosystems, Foster City, Calif.).

A non-limiting list of other applicable polymer substrates includes: (1) Particles based upon copolymers of dimethylacrylamide cross-linked with N,N′-bisacryloylethylenediamine, including a known amount of N-tertbutoxycarbonyl-beta-alanyl-N′-acryloylhexamethylenediamine. Several spacer molecules may be added via the beta alanyl group, followed thereafter by the amino acid residue subunits. Also, the beta alanyl-containing monomer can be replaced with an acryloyl sarcosine monomer during polymerization to form resin beads. The polymerization is followed by reaction of the beads with ethylenediamine to form resin particles that contain primary amines as the covalently linked functionality. The polyacrylamide-based supports are relatively more hydrophilic than are the polystyrene-based supports and are usually used with polar aprotic solvents including dimethylformamide, dimethylacetamide, N-methylpyrrolidone, and the like.

(2) A second group of substrates is based on silica-containing particles such as porous glass beads and silica gel, including the reaction product of trichloro-[3-(4-chloromethyl)phenyl]propylsilane and porous glass beads (commercially available as PORASIL E® from Waters Corp., Milford, Mass.) and a mono ester of 1,4-dihydroxymethylbenzene and silica (commercially available as BIOPAK® from Waters Corp., Milford, Mass.).

(3) A third general type of useful solid substrates can be termed composites in that they contain two major ingredients: a resin and another material that is also substantially inert to the reaction conditions employed. One exemplary composite utilizes glass particles coated with a hydrophobic, cross-linked styrene polymer containing reactive chloromethyl groups. Another exemplary composite contains a core of fluorinated ethylene polymer onto which has been grafted polystyrene.

(4) Contiguous solid supports, such as cotton sheets and hydroxypropylacrylate-coated polypropylene membranes also are suited for use as the substrate. Particularly preferred is the polyethylene/polystyrene (PEPS) matrix, which also is commonly used in the solid phase synthesis of PNAs. The PEPS matrix comprises a polyethylene (PE) film with pendant long-chain polystyrene (PS) grafts. The PEPS film may be fashioned in the form of discrete, labeled sheets, each serving as an individual reaction compartment. Alternative geometries of the PEPS polymer such as, for example, non-woven felt, knitted net, sticks, and microwellplates also are appropriate.

(5) Acrylic acid-grafted polyethylene-rods and 96-microtiter wells also are appropriate matrices. Sometimes, this method may only be applicable on a microgram scale.

Any appropriate solvent likewise may be utilized in the present invention to suspend the substrate, as will be appreciated by one skilled in the art, using the guidelines provided herein. The most commonly used solvents include N,N-dimethylformamide (DMF), dichloromethane (DCM), N-methyl-2-pyrrolidinone (NMP), and mixtures and combination thereof. Other exemplary solvents include water, dimethyl sulfoxide (DMSO), methanol (MeOH), dioxane, dimethylacetamide (DMA), ethyl acetate, and mixtures and combinations thereof. The solvent may preferably be chosen to correspond with the polymer substrate. Additionally, it may be desirable to swell the polymer substrate in a solvent and then exchange the solvent. In a preferred embodiment, (methyl-benzhydryl) amine polystyrene resin is swelled in DCM and subsequently exchanged out for DMF.

The substrate and solvent may be physically contained in a variety of different manners, as will be appreciated by one skilled in the art using the guidelines contained herein. For example, the substrate may be contained in a “tea bag” that is submersed in the solvent. Other alternatives include, but are not limited to, two different supports with different densities, combining reaction vessels via a manifold, multicolumn supports, and the use of cellulose paper. Any number of applicable glassware setups also may be used, as will be appreciated by one skilled in the art.

The targeting moiety may be attached to the substrate in any applicable fashion to form a targeting moiety—substrate component. Attaching schemes used in the solid phase synthesis of polypeptides, for example, are preferred methods for attaching the targeting moiety to the substrate. For example, anchoring linkages may be used to attach the targeting moiety to the substrate. Exemplary anchoring linkages include the chloromethyl, aminomethyl, and benzhydrylamino functionalities. These are the most widely applied functionalities in SPPS. Other reactive functionalities serving as anchoring linkages include 4-methylbenzhydrylamino and 4-methoxybenzhydrylamino.

Aminomethyl is a preferred anchoring linkage because aminomethyl is particularly advantageous with respect to the incorporation of “spacer” or “handle” groups. Representative spacer- or handle-forming bifunctional reagents include 4-(haloalkyl)aryl-lower alkanoic acids such as 4-(bromomethyl)phenylacetic acid, Boc-aminoacyl-4-(oxymethyl)aryl-lower alkanoic acids such as Boc-aminoacyl-4-(oxymethyl)phenylacetic acid, N-Boc-p-acylbenzhydrylamines such as N-Boc-p-glutaroylbenzhydrylamine, N-Boc-4′-lower alkyl-p-acylbenzhydrylamines such as N-Boc-4′-methyl-p-glutaroylbenzhydrylamine, N-Boc-4′-lower alkoxy-p-acylbenzhydrylamines such as N-Boc-4′-methoxy-p-glutaroyl-benzhydrylamine, and 4-hydroxymethylphenoxyacetic acid. A preferred spacer group which is often used for the solid phase synthesis of peptides is phenylacetamidomethyl (PAM). PAM is advantageous because of its stability towards the BOC-amino deprotection reagent trifluoroacetic acid (TFA), which may be used in accordance with the present invention.

An alternative strategy for the introduction of spacer or handle groups that may offer more control over attachment of the targeting moiety to the substrate is the “preformed handle” strategy. In the preformed handle strategy, spacer or handle groups of the same type as described herein are reacted with the targeting moiety that is to be attached to the substrate. Thus, in those cases in which a spacer or handle group is desirable, the targeting moiety may either be coupled to the free reactive end of a spacer group that has already been bound to an initially introduced functionality (for example, an aminomethyl group) or can be reacted with the spacer-forming reagent and then reacted with the initially introduced functionality. In both cases, the targeting moiety-spacer-reactive functionality compound subsequently attaches to the polymer substrate. Other useful anchoring schemes include the “multidetachable” resins that provide more than one mode of release and thereby allow more flexibility in synthetic design.

One skilled in the art will appreciate that any appropriate anchoring scheme comprising, for example, anchoring linkages and spacer- or handle-forming groups may be employed in the present invention to attach the targeting moiety to the substrate, according to the guidelines provided herein. The attachment of an amino acid targeting moiety to a substrate is exemplarily illustrated in (2) of Synthesis I.

If the targeting moiety contains reactive groups, for example amino groups located at the terminus and side chains of the targeting moiety, it may be preferable to protect the reactive groups with protecting groups during attachment of the targeting moiety to the polymer substrate. Hence, (2) of Synthesis I denotes R′, the protected form of the side chain group R, and NH′, the protected form of the terminal amino group NH₂. Other reactive groups of the targeting moiety that also may be protected during attachment to the substrate include, but are not limited to, phosphate and carboxyl groups.

Amino groups, for example the terminal amino group and amino groups located in the side chains of the amino acid exemplarily depicted in Synthesis I, may be protected with any applicable amino protecting groups. The two most common protecting schemes for amino groups use either the tert-butyloxycarbonyl (Boc) group or the 9-fluorenylmethyloxycarbonyl (Fmoc) group. Other useful amino protecting groups include, but are not limited to, adamantyloxycarbonyl (Adoc), 2-(4-Biphenyl)isopropyloxycarbonyl (Bpoc), Mcb, Bic, o-nitophenylsulfenyl (Nps), dithiasuccinoyl (Dts), methoxy trityl (Mtt), and benzhydryloxycarbonyl (Bhoc). In general, any amino protecting group which largely fulfills one or more of the following requirements may be utilized in accordance with the present invention: (1) stability to mild acids (not significantly attacked by carboxyl groups); (2) stability to mild bases or nucleophiles (not significantly attacked by the amino group in question); (3) resistance to acylation (not significantly attacked by activated amino acids); (4) is close to being quantitatively removable without serious side reactions; and (5) preserves the optical integrity, if any, of the targeting moiety.

It may be desirable to preferentially remove specific protecting groups without affecting other protecting groups. For example, it may be desirable to preferentially remove the protecting group of the terminal amino group, NH′, of the amino acid exemplarily illustrated in (2) of Synthesis I without removing the protecting group of the side chain, R′. Therefore, complementary protecting groups that are removed by different reaction conditions may be chosen to protect different reactive groups. For example, a protecting group that is removed by acidic conditions may protect an amino group in a side chain while a protecting group that is removed by basic conditions may protect a terminal amino group. Alternatively, a protecting group that is sensitive to slightly acidic conditions may protect one reactive group while a protecting group that is sensitive only to strongly acidic conditions protects another reactive group. One skilled in the art will appreciate the wide range of protecting groups and protecting schemes that may be utilized in the present invention, in accordance with the guidelines presented herein.

In a preferred embodiment, the targeting moiety—substrate component may be linked with one or more additional monomeric units before conjugation with one or more complexing agents. In a further preferred embodiment, the additional monomeric units may be selected from natural amino acids, unnatural amino acids, peptides, peptide nucleic acids, nucleotides, and analogs and derivatives thereof. One skilled in the art will appreciate that other possible additional monomeric units also may be used in accordance with the present invention, following the guidelines provided herein.

For example, an amino acid based targeting moiety—substrate component may be linked with additional amino acid monomers, as is exemplarily illustrated in (3) of Synthesis I, where the linking of the two amino acids is accomplished by a condensation reaction between the α-carbonyl of the additional amino acid and the terminal amino group of the amino acid attached to the substrate.

Reactive groups of the targeting moiety—substrate component that may have been protected during attachment of the targeting moiety to the substrate may be deprotected to enable the underlying functionality during linking with the one or more additional monomeric units. This may be preferred, for example, if the deprotected reactive group is to be involved in the linking scheme. For example, in Synthesis I the terminal amino group of the first amino acid attached to the substrate was protected during attachment to the substrate in (2) but may be deprotected during linking with the additional amino acid depicted in (3) in order to enable the terminal amino group to participate in the condensation reaction with the carboxyl group of the additional amino acid. The deprotection of reactive groups of the targeting moiety—substrate component may be in any applicable manner. For example, acid or base washes may be used to remove amino protecting groups. The Fmoc amino protecting group may be removed with a basic solution such as 20% piperidine in N,N-dimethyl formamide (DMF). The Boc amino protecting group may be removed with an acidic solution such as hydrofluoric acid (HF) or trifluoroacetic acid (TFA). One skilled in the art will appreciate that the process for deprotection may be chosen according to the protecting group employed.

In a preferred embodiment, the additional monomeric units each has only one free amino-reactive group. This may be advantageous so as to link the targeting moiety—substrate component and the additional monomeric units at a single selected amino-reactive group on each additional monomeric unit. This may be accomplished by protecting amino-reactive groups of the additional monomeric units that are not intended to be involved in the linking scheme. One skilled in the art will appreciate the protecting groups that may be utilized to protect the amino-reactive groups of the additional monomeric units.

Also, more than one protecting group may be utilized. It may be preferable, for example, to protect certain reactive groups, such as amino groups located in side chains, if any, of the additional monomeric units, in such a manner so that the protecting groups may be selectively removed at a later time. In such a situation, it may be advantageous to use more than one protecting group. In Synthesis I, for example, the benzhydryloxycarbonyl (Bhoc) protecting group preferably is utilized to protect amino groups in the side chain of the additional amino acid, R′ in (3), during coupling to the amino acid attached to the substrate. A different protecting group might be chosen to protect the terminal amino group of the additional amino acid, NH′ in (3). In this way, one of the protecting groups may be removed at a later time without removing the other protecting group.

Other reactive groups of the additional monomeric units, such as phosphate and carboxylic groups, also may be protected during linking to the targeting species—substrate component. One skilled in the art will appreciate the myriad protecting groups that may be chosen to protect their respective reactive groups.

In a preferred embodiment, the one or more additional monomeric units are linked to the targeting moiety via a reaction between a free amino group of the targeting moiety—substrate component and an amino-reactive group of the additional monomeric units. Each of the additional monomeric units also may comprise one or more protected amino groups besides the amino-reactive group. Following linking with the targeting moiety—substrate component, an amino group of the additional monomeric unit (now part of the targeting moiety) may be deprotected in order to participate in linking with amino-reactive groups of subsequent additional monomeric units. In this fashion, a series of additional monomeric units may be linked to each other and the targeting moiety via reactions between free amino groups and amino-reactive groups.

In a preferred embodiment, the amino-reactive groups of the additional monomeric units are carboxyl groups. As described herein, amino groups and carboxyl groups may participate in condensation reactions with each other. The result of a condensation reaction between an amino acid based targeting moiety and an amino acid based additional monomeric unit is exemplarily illustrated in (4) of Synthesis I. The linking scheme may be repeated many times to produce a polymeric targeting moiety—substrate component, such as the oligopeptide based targeting moiety—substrate component exemplarily illustrated in (5) of Synthesis I. In a preferred embodiment, the condensation reaction is assisted by activating the carbonyl group.

Activation of the carbonyl group may be accomplished, for example, by forming the active ester. Formation of an active ester is often accomplished by the addition of a benzotriazole-based compound. Exemplary benzotriazole-based compounds that may be used to form an active ester include, but are not limited to, 1-Hydroxybenzotriazole (HOBt), 1-hydroxy-7-azabenzotriazole (HOAt), 1-H-Benzotriazolium-1-[bis(dimethylamino)methylene]-5-chloro-tetrafluoroborate(1-),3-oxide (TBTU), 1-[bis(dimethylamino)methylene]-hexafluorophosphate(1-), and 3-oxide O-(Benzotriazol-1-yl)-N,N,N′,N′ tetramethyluronium hexafluorophosphate (HBTU). Other activating agents include, but are not limited to, 1-[bis(dimethylamino)methylene]-5-chloro-hexafluorophosphate (1-),3-oxide (HCTU), O-(Cyano(ethoxycarbonyl)methylenamino)-1,1,3,3-tetramethyluronium tetrafluoroborate (TOTU), and 2-(1H-azabenzotriazole-1-yl)-1,1,3,3-tetramethyluronium hexafluorophosphate (HATU). Activating agents may be accompanied by a base such as N,N-diisopropylethylamine (DIEA).

In another preferred embodiment, the condensation reaction is assisted by the addition of a condensation reagent. Exemplary condensation reagents include carbodiimides such as dicyclohexylcarbodiimide (DCC) and diisoproplycarbodiimide (DIC), phosphonium salts, uronium salts, and derivatives thereof. The carbonyl group also may be activated by forming an acid halide. This, however, may not be an ideal method because of the possibility of intramolecular reaction. Some acid fluorides, however, have proven to be less susceptible to intramolecular reactions. Yet another applicable method of activating the carbonyl group is the formation of an anhydride. One skilled in the art will appreciate the many alternatives wherein a condensation reaction may be facilitated.

The complexing agent used in the present invention may be any applicable complexing agent, in accordance with the limitations and guidelines provided herein. In a preferred embodiment, the complexing agent is a DOTA or TETA compound of the formula (II): 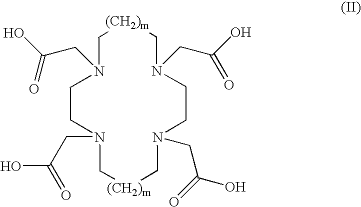
where m is 1 or 2.

One skilled in the art will appreciate that other complexing agents, for example other macrocyclic polyaza compounds and other derivatives and analogs of various complexing agents may be conjugated to the targeting moiety—substrate component. A preferred derivative of a complexing agent is a complexing agent wherein reactive groups, especially amino-reactive groups, that are not intended to be involved in the conjugation of the complexing agent to the targeting moiety—substrate component are protected in order to prevent unwanted reactions. For example, a preferred derivative of the complexing agents DOTA and TETA is the tri-protected form of the compound of formula (II), which is shown below as formula (III): 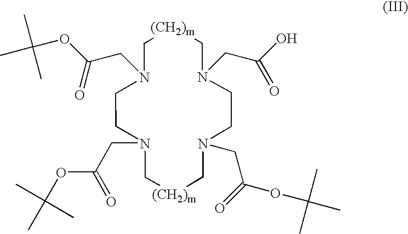
where m is 1 or 2. The compound of formula (III) may be conjugated to the targeting moiety—substrate component and deprotected at a later time so as to enable its full functionality as a complexing agent. The tri-protected compound of formula (III) may be advantageous because only one amino-reactive group is free to participate in conjugation to the targeting moiety—substrate complex. This may help avoid over-substitution of the compound.

The choice of complexing agents may be governed, for example, by the affinity of the complexing agents to desired radioactive elements to be complexed with the complexing agents at a later time. The choice also may be affected by a desired biocompatibility of the complexing agents. Molecular geometry and cost are other exemplary factors that may be important in choosing the one or more complexing agents to be conjugated to the targeting moiety—substrate component.

Applicable complexing agents include, but are not limited to, diethylenetriamine-pentaacetic acid (“DTPA”); 1,4,7,10-tetraazacyclododecane-N,N′,N″,N′″-tetraacetic acid (“DOTA”); p-isothiocyanatobenzyl-1,4,7,10-tetraazacyclododecane-1,4,7,10-tetraacetic acid (“p-SCN-Bz-DOTA”); 1,4,7,10-tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (“NOTA”); 1,4,8,11-tetraazacyclotetradecane-N, N′,N″,N′″-tetraacetic acid (“TETA”); triethylene tetraamine hexaacetic acid (“TTHA”); trans-1,2-diaminohexane tetraacetic acid (“CYDTA”); 1,4,7,10-tetraazacyclododecane-1-(2-hydroxypropyl)4,7,10-triacetic acid (“HP-DO3A”); trans-cyclohexane-diamine tetraacetic acid (“CDTA”); trans(1,2)-cyclohexane diethylene triamine pentaacetic acid (“CDTPA”); 1-oxa-4,7,10-triazacyclododecane-N,N′,N″-triacetic acid (“OTTA”); 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives and analogs thereof, particularly protected forms of the compounds.

One or more complexing agents may be conjugated to the targeting moiety—substrate component at one or more free amino groups of the targeting moiety—substrate component to form a complexing targeting moiety—substrate component. The free amino groups may be located, for example, at a terminus of the targeting moiety—substrate component, a side chain of the targeting moiety—substrate component, the polymeric backbone of the targeting moiety—substrate component, or elsewhere. Conjugation of a complexing agent with a polypeptide based targeting moiety—substrate component at a terminal free amino group is exemplarily illustrated in (6) of Synthesis I. Because it may be desirable to protect some of the reactive groups of the complexing agent during conjugation to the targeting moiety—substrate component, the protected form of the complexing agent, CX′, is illustrated in (6) of Synthesis I. If needed, the amino group of the targeting moiety—substrate component to which conjugation will occur may be deprotected prior to conjugation.

If desired, the complexing agent may be activated to facilitate conjugation to the targeting moiety—substrate component. Activation using a carboxyl activating group, for example, may facilitate conjugation of the complexing agent to the targeting moiety—substrate component via a condensation reaction between a carboxyl group of the complexing agent and one or more free amino groups of the targeting moiety—substrate component. For example, it is preferred that a carboxyl group of the compound of formula (III) be activated with HATU in order to react with one or more free amino groups of the targeting moiety—substrate component. Other activating agents include, but are not limited to, HOBt, HOAt, TBTU, HBTU, HCTU, and TOTU. Activating agents may be accompanied by a base such as DIEA. In another exemplary activating method, a fluoride of the complexing agent is formed. In yet another exemplary activating method, an anhydride of the complexing agent is formed. In still another exemplary method for affecting the conjugation of the complexing agent with one or more free amino groups of the targeting moiety—substrate component, a condensation reagent such as the carbodiimides dicyclohexylcarbodiimide (DCC) and diisoproplycarbodiimide (DIC), phosphonium salts, or uronium salts are used. One skilled in the art will appreciate the other activating agents that may be used in accordance with the present invention to affect a condensation reaction between an amino group of the targeting moiety—substrate component and a carboxyl group of the complexing agent.

Though (6) of Synthesis I exemplarily illustrates conjugation of the complexing agent to the terminal amino group of a polypeptide chain attached to the polymer substrate, it should be understood that the complexing agent may alternatively be conjugated at one or more free amino groups located at one or more side groups, the backbone, or elsewhere in the targeting moiety—substrate component. For example, if the targeting moiety—substrate component contains a side group that is the side group of the lysine amino acid (—(CH₂)₄NH₂), then the complexing agent may be conjugated to the amino group at the end of the lysine based side group of the targeting moiety—substrate component.

One skilled in the art will recognize still other methods wherein the complexing agent may be conjugated at one or more free amino groups of the targeting moiety—substrate component, in accordance with the guidelines provided herein.

In another preferred embodiment, one or more additional monomeric units may be linked to the complexing targeting moiety—substrate component. In this way, the targeting moiety portion of the complexing targeting moiety—substrate component may be modified even after conjugation with the complexing agent. Though such an embodiment is not exemplarily illustrated in Synthesis I, it should be understood that additional monomeric units may be linked to the polypeptide chain conjugated to the complexing agent illustrated in (6). The additional monomeric units may be linked to the complexing targeting moiety—substrate component in the same fashion as the linking of additional monomeric units to the targeting—moiety substrate component, as described herein. A preferred method for linking additional monomeric units to the complexing targeting moiety—substrate component, for example, is through condensation reactions between an activated carbonyl group of the additional monomeric units and a free amino group of the complexing targeting moiety—substrate component.

Any applicable additional monomeric unit may be linked to the complexing targeting moiety—substrate component. For example, the additional monomeric units may be independently selected from natural amino acids, unnatural amino acids, peptides, peptide nucleic acids, nucleotides, and analogs and derivatives thereof. In a preferred embodiment, the additional monomeric units each has only one free amino-reactive group prior to linking to the targeting moiety—substrate component.

The complexing targeting moiety may be cleaved from the substrate using any applicable process, following the guidelines provided herein. Cleavage of the complexing targeting moiety, for example, may be accomplished similar to the cleavage of a polypeptide from the polymer substrate, as is exemplarily illustrated in (8) of Synthesis I. In a preferred embodiment of the present invention, the complexing targeting moiety is cleaved from the substrate using an acidic solution. For example, a solution of trifluoroacetic acid (TFA) may be used to cleave the complexing targeting moiety from the substrate. A solution of at least 82% TFA in phenol, thioanisol, water, ethanedithiol, and triisopropylsilane, for example, also is appropriate. Alternatively, other acid solutions, for example hydrofluoric acid (HF) and sulfonic acids such as trifluoromethanesulfonic acid and methanesulfonic acid, may be used. In yet another example, the complexing targeting moiety is cleaved from the substrate using a mixture of TFA and 20% m-cresol; the substrate may be filtered using glass wool and rinsed with TFA; and the complexing targeting moiety may be precipitated using cold ether and a centrifuge. Basic solutions such as an ammonia solution are also applicable. One skilled in the art will recognize other methods by which the complexing targeting moiety may be cleaved from the substrate.

The complexing targeting moiety may be deprotected following conjugation, as is exemplarily illustrated in (7) of Synthesis 1. The deprotection process, as will be appreciated by one skilled in the art, will be tailored to the particular protecting groups chosen to protect the various reactive groups of the complexing targeting moiety. The complexing targeting moiety may be deprotected, for example, by rinsing the complexing targeting moiety in a basic solution or an acidic solution.

The acidic deprotection method may produce very reactive carbocations that may lead to alkylation and acylation of sensitive residues in the complexing targeting moiety. Such undesirable side-reactions may be partly avoided by the addition of scavengers such as anisole, phenol, dimethyl sulfide, and mercaptoethanol. The sulfide-assisted acidolytic S^{N}2 deprotection method, which removes the precursors of harmful carbocations to form inert sulfonium salts, also may be employed during cleavage of the complexing targeting moiety from the polymer substrate, either solely or in combination with other methods to suppress carbocation-induced side reactions. Other methods used for deprotection include, for example, rinsing the substrate with a solution of base-catalyzed alcoholycis, ammonolysis, hydrazinolysis, hydrogenolysis, and photolysis. All of these and other applicable deprotection methods may be utilized in accordance with the present invention.

In a preferred embodiment, the complexing targeting moiety may be deprotected and cleaved from the substrate concurrently.

At various times during the preparation of the targeting moiety—substrate component and conjugation with the complexing agent, it may be desirable to wash the products of a reaction in order to remove unwanted by-products, reagents, solvents, and other contaminants from the solution in which the reaction took place. During washing, the targeting moiety—substrate component or complexing targeting moiety—substrate component may be subjected to solvent rinses that help to wash away contaminants. The targeting moiety—substrate component or complexing targeting moiety—substrate component also may be subjected to filtering cycles that remove the substrate and attached compounds from the solution by filtering the solution using an appropriate medium. For example, cloth, paper, or ceramic filters may be used to remove the substrate from the solution. Additionally the targeting moiety—substrate component or complexing targeting moiety—substrate component may be dried, for example, by placing it under vacuum, air-drying, blowing nitrogen or another gas across the substrate, or in any other applicable manner. Drying may be useful, for example, in removing an unwanted solvent that may be difficult to remove using a washing sequence.

In another embodiment of the present invention, the targeting moiety is one or more monomeric units of the formula (IV): 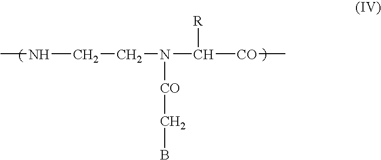
where B is a heterocyclic base independently selected from adenine, guanine, cytosine, thymine, and uracil; and R is independently selected from hydrogen and the side groups covalently bonded to α-carbons of the naturally occurring α-amino acids. In another preferred embodiment, the additional monomeric units that may be linked to either the complexing targeting moiety—substrate component or the targeting moiety—substrate component are also amino acids of formula IV.

Systematic linking of additional monomeric units of formula IV to a targeting moiety of one or more monomeric units of formula IV may yield a peptide nucleic acid of the formula (V): 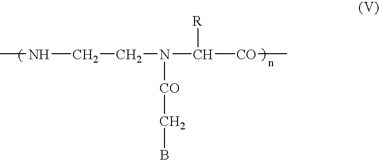
where B is a heterocyclic base independently selected from adenine, guanine, cytosine, thymine, and uracil; R is independently selected from hydrogen and the side groups covalently bonded to α-carbons of the naturally occurring α-amino acids; and n is an integer in a range of from about 4 to about 20, inclusive.

In another embodiment of the present invention, the targeting moiety is a peptide nucleic acid of formula V. Additional monomeric units such as nucleotide units may be linked to the targeting moiety either before or after conjugation with the complexing agent. For example, adenine, guanine, cytosine, thymine, and uracil may be linked to the targeting moiety via a Dmt-protected N-(2-hydroxyalkyl)glycine building block. The building block may be coupled to the terminal amino group of the PNA based targeting moiety. The Dmt protecting group may be removed from the hydroxyl group of the building block using 3% trichloroacetic acid (TCA) in dichloromethane (DCM). A standard nucleoside-3′-phosphoramidite may be coupled to the deprotected hydroxyl group of the building block. Additional monomeric units, preferably additional nucleotide units, then may be linked to the nucleoside-3′-phosphoramidite to further elaborate the targeting moiety—substrate component. This may result in a targeting moiety that is PNA-DNA chimera.

As described herein, the targeting moiety—substrate component may be elaborated by linking with additional monomeric units either before or after conjugation with the complexing agent. The complexing targeting moiety then may be cleaved from the substrate. Therefore, a wide variety of radiopharmaceuticals may be synthesized by conjugation of one or more complexing agents with a targeting moiety in accordance with the present invention. For example, another exemplary embodiment provides a radiopharmaceutical of the formula (VII): 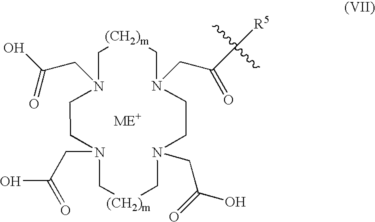
where m is 1 or 2; ME⁺ is a radioactive metal ion; and R⁵ is a targeting moiety comprising at least one monomeric unit from the group of natural amino acids, unnatural amino acids, peptides, peptide nucleic acids, nucleotides, and analogs and derivatives thereof.

For example, R⁵ may be a targeting moiety of the formula (VIII): 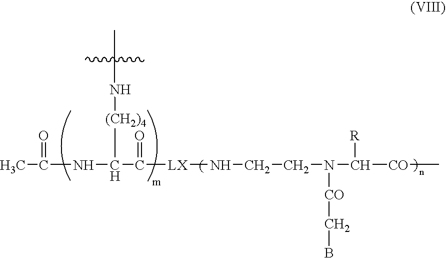
where B is a heterocyclic base independently selected from adenine, guanine, cytosine, thymine, and uracil; m is an integer in the range of from about 1 to about 600; n is an integer in the range of from about 4 to about 20, inclusive; R is independently selected from hydrogen and the side groups covalently bonded to α-carbons of the naturally occurring α-amino acids; and LX is selected from a direct bond and a linker having the formula (—CH₂—CH₂—O—)ₚ, where p is an integer in the range of from about 1 to about 50, inclusive. Just one, more than one, or all of the “m” number of lysine units as shown in formula VIII may be conjugated to a complexing agent.

There are several exemplary methods suitable to synthesize the compound of formula VIII in accordance with the present invention. In a first example, the targeting moiety may comprise “m” (from about 1 to about 600) monomeric units of lysine. The targeting moiety is attached to the substrate and the targeting moiety—substrate component may be conjugated with one or more complexing agent and then linked to “n” (from about 1 to about 20) additional monomeric units of formula IV before cleaving from the substrate.

In a second example, the targeting moiety may be a single monomeric unit of lysine. The targeting moiety is attached to the substrate and the targeting moiety—substrate component may be linked with “m” (from about 1 to about 600) additional monomeric units of lysine and then “n” (from about 1 to about 20) additional monomeric units of formula IV. The targeting moiety—substrate component then may be conjugated with one or more complexing agents.

In a third example, the targeting moiety may be a single monomeric lysine unit. The targeting moiety is attached to the substrate and the targeting moiety—substrate component may be conjugated with the complexing agent and then linked with one or more additional monomeric units such as “m” (from about 1 to about 600) lysine units and “n” (from about 1 to about 20) units of the compound of formula IV. Finally, the complexing targeting moiety may be cleaved from the substrate.

One skilled in the art will appreciate that there are still other methods to synthesize the compound of formula VIII in accordance with the present invention.

In another example, R⁵ may be a targeting moiety of the formula (IX): 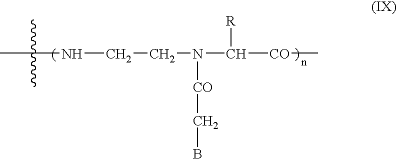
where B is a heterocyclic base independently selected from adenine, guanine, cytosine, thymine, and uracil; R is independently selected from hydrogen and the side groups covalently bonded to α-carbons of the naturally occurring α-amino acids; and n is an integer in a range of from about 4 to about 20, inclusive.

There are several methods suitable to synthesize the compound of formula IX in accordance with the present invention. For example, the targeting moiety may be a single monomeric unit of formula IV. The targeting moiety is attached to the substrate and the targeting moiety—substrate component may linked to additional monomeric units of formula IV before conjugating with the complexing agent and cleaving from the substrate.

One skilled in the art will appreciate that there are still other methods to synthesize the compound of formula IX in accordance with the present invention.

In another example, R⁵ may be a targeting moiety of the formula (X): 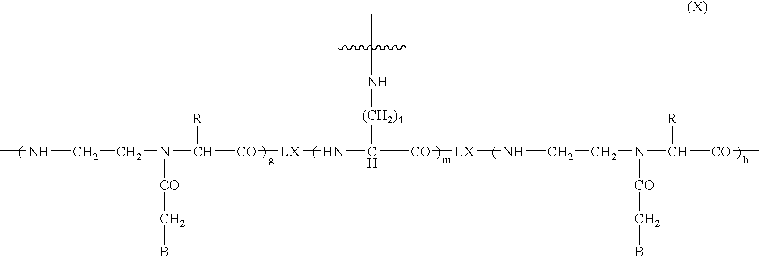
where B is a heterocyclic base independently selected from adenine, guanine, cytosine, thymine, and uracil; n is an integer in the range of from about 4 to about 20, inclusive; R is independently selected from hydrogen and the side groups covalently bonded to α-carbons of the naturally occurring α-amino acids; g is an integer in the range of from about 1 to about 20, inclusive; h is an integer in the range of from about 1 to about 20, inclusive; LX is selected from a direct bond and a linker having the formula (—CH₂—CH₂—O—)ₚ, where p is an integer in the range of from about 1 to about 50, inclusive; and m is an integer in the range of from about 1 to about 600, inclusive. Just one, more than one, or all of the “m” number of lysine units as shown in formula IX may be conjugated to a complexing agent.

There are several methods suitable to synthesize the compound of formula X in accordance with the present invention. For example, the targeting moiety may be “g” (from about 1 to about 20) monomeric units of formula IV. The targeting moiety is attached to the substrate and the targeting moiety—substrate component may be linked to “m” (from about 1 to about 600) additional lysine monomeric units via a linker and then linked via another linker to “h” (from about 1 to about 20) additional monomeric units of formula IV. The targeting moiety—substrate component then may be conjugated with one or more complexing agents and cleaved from the substrate.

In another example, the targeting moiety may be “g” (from about 1 to about 20) monomeric units of formula IV. The targeting moiety is attached to the substrate and the targeting moiety—substrate component may be linked to “m” (from about 1 to about 600) additional lysine monomeric units via a linker and then conjugated with one or more complexing agents at the “m” number of additional lysine monomeric units. The complexing targeting moiety—substrate component then may be linked via a linker to “h” (from about 1 to about 20) additional monomeric units of formula IV. The complexing targeting agent finally may be cleaved from the substrate.

One skilled in the art will appreciate that there are still other methods to synthesize the compound of formula X in accordance with the present invention.

In another embodiment of the present invention, the targeting moiety may be one or more nucleotides, nucleotide analogs, or nucleotide derivatives. For example, the targeting moiety may be a single nucleotide base such as adenine, guanine, cytosine, thymine, or uracil. These five bases are the bases found in DNA and RNA and each comprise a 5′-hydroxyl group, a phosphorus linkage, and other reactive groups. In general, the nucleotide base may be attached to a substrate. During attachment, the reactive groups such as the 5′-hydroxyl group may be protected. The 5′-hydroxyl group, for example, may be protected with the dimethoxytrityl (DMT). Preferred substrates for attachment of nucleotides includes controlled-pore glass (CPG) and TentaGel® (commercially available from Rapp Polymere Gmbh, Tubingen, Germany).

Following attachment to the substrate, additional monomeric units such as natural amino acids, unnatural amino acids, peptides, peptide nucleic acids, nucleotides, and analogs and derivatives thereof may be linked to the nucleotide based targeting moiety—substrate component. Preferred additional monomeric units are nucleotide bases. Linking with additional nucleotide bases may be accomplished, for example, by activating the phosphorus linkage of the additional nucleotide base and reacting it with the deprotected 5′-hydroxyl group of the nucleotide based targeting moiety—substrate component. Deprotection of the 5′-hydroxyl group may be accomplished by removing the DMT protecting group with an acidic solution such as dichloroacetic acid (DCA) or trichloroacetic acid (TCA) in dichloromethane (DCM). The phosphorus linkage of the additional nucleotide base may be activated, for example, with tetraazole. The free hydroxyl group and activated phosphorus may react to form an unstable phosphite linkage. 5′-hydroxyl groups that are unreacted may be capped or otherwise protected to prevent their reaction in subsequent synthetic steps. For example, unreacted 5′-hydroxyl groups may be capped by acetylation with acetic anhydride and N-methylimidazole. Following capping of unreacted 5′-hydroxyl groups, the unstable phosphite linkages may be oxidized to form stable phosphate linkages. This may be accomplished, for example, by addition of a solution of dilute iodine in water, pyridine, and tetrahydrofuran.

By repeating the hydroxyl-phosphorus linking process, many additional nucleotide units may be linked to the targeting moiety—substrate component. One skilled in the art will recognize that different linking schemes may be utilized in order to attach other additional monomeric units, such as natural amino acids, unnatural amino acids, peptides, peptide nucleic acids, nucleotides, and analogs and derivatives thereof to the nucleotide based targeting moiety—substrate component or the nucleotide based complexing targeting moiety—substrate component.

Another preferred additional monomeric unit that may be linked to the nucleotide based targeting moiety—substrate component is a PNA. This may be accomplished by attaching a 5′-N-Mmt-5′-amino-2′,5′-dideoxynucleoside-3′-phosphoramidite linker to the last nucleotide base in the targeting moiety—substrate component. The 5′-terminal N-Mmt group may be removed with TCA, to which the additional PNA monomeric units may be linked via reaction with an amino-reactive group of the PNA such as the carboxyl groups. Then, other additional monomeric units, preferably additional PNA monomeric units, may be linked to the terminal PNA unit of the targeting moiety—substrate component. This may result in a targeting moiety that is a DNA-PNA chimera.

At any time during or before modification of the targeting moiety by linking with additional monomeric units, one or more complexing agents such as those described herein may be conjugated to one or more free amino groups of the targeting moiety—substrate component. It may be necessary to link one or more lysine groups, lysine analogs, or lysine derivatives as additional monomeric units to the targeting moiety—substrate component in order to introduce free amino groups to the targeting moiety. One or more complexing agents may be conjugated to the targeting moiety—substrate component via reactions between the free amino groups of the targeting moiety—substrate component and amino-reactive groups of the complexing agents. For example, a condensation reaction between an activated hydroxyl group of the complexing agent and a free amino group of the targeting moiety—substrate component may link the two compounds, as described herein. One skilled in the art will appreciate the myriad other ways in which conjugation of the targeting moiety—substrate component and complexing agent may be accomplished, in accordance with the guidelines herein.

Once a desired complexing targeting moiety—substrate component has been synthesized, the complexing targeting moiety may be cleaved from the substrate. Additionally, protecting groups on the complexing targeting moiety may preferably be removed at the same time. One skilled in the art will appreciate how this is to be done, in accordance with the guidelines herein.

The complexing targeting moieties of the present invention may be complexed with a radioactive element in preparation for use as a radiopharmaceutical. The complexing targeting moiety may be radiolabeled at any time following conjugation to the targeting moiety—substrate component. Alternatively, the complexing agent may be radiolabeled before conjugation to the targeting moiety—substrate component. Because the reaction conditions involved in the optional attachment of one or more additional monomeric units to the complexing targeting moiety—substrate component and cleavage of the complexing targeting moiety from the substrate may affect the stability of the radiolabeled complexing targeting moiety, it is preferred that the radioactive element be complexed to the complexing targeting moiety after the complexing targeting moiety has been synthesized, cleaved from the substrate, and optionally purified.

The radiolabeling process may be performed in any appropriate manner as will be appreciated by one skilled in the art using the guidelines provided herein. For example, the complexing targeting moiety may be contacted with an ion transfer material having the radioactive metal ion bound thereto and having a binding affinity for the radioactive metal less than the binding affinity for the radioactive metal ion of the complexing targeting moiety. Prior to contacting, the complexing portion of complexing targeting moiety is either uncomplexed or is complexed with a second metal having a binding affinity with the complexing portion less than the binding affinity of the radioactive metal ion. Upon contact with the ion transfer material, the radioactive metal ion transfers from the material to the complexing targeting moiety. If the complexing targeting moiety is already complexed to a metal ion, the metal ion is exchanged for the radioactive metal ion. The radiolabeled complexing targeting moiety is subsequently separated from the ion transfer material and purified.

In another exemplary radiolabeling process, the complexing targeting moiety may be dissolved in a buffered aqueous solution of the radionuclide. The pH may be selected to optimize conditions for complexation of the radioactive element with the complexing targeting moiety. The reaction mixture temperature also may be adjusted to promote complexation of the radionuclide with the complexing targeting moiety. After a period of time, the solution is quenched by the addition of an anionic quenching chelate such as diethylenetriaminepentaacetic acid (DTPA) and the reaction mixture then is purified.

The radioactive metal ion complexed with the complexing targeting moiety may be from any appropriate metallic radioisotope including, but not limited to, actinium-225, astatine-211, iodine-120, iodine-123, iodine-124, iodine-125, iodine-126, iodine-131, iodine-133, bismuth-212, arsenic-72, bromine-75, bromine-76, bromine-77, indium-110, indium-111, indium-113m, gallium-67, gallium-68, strontium-83, zirconium-89, ruthenium-95, ruthenium-97, ruthenium-103, ruthenium-105, mercury-107, mercury-203, rhenium-186, rhenium-188, tellurium-121 m, tellurium-122m, tellurium-125m, thulium-165, thulium-167, thulium-168, technetium-94m, technetium-99m, fluorine-18, silver-111, platinum-197, palladium-109, copper-62, copper-64, copper-67, phosphorus-32, phosphorus-33, yttrium-86, yttrium-90, scandium-47, samarium-153, lutetium-177, rhodium-105, praseodymium-142, praseodymium-143, terbium-161, holmium-166, gold-199, cobalt-57, cobalt-58, chromium-51, iron-59, selenium-75, thallium-201, and ytterbium-169.

Additionally, methods to stabilize the radiolabeled complexing targeting moiety in order to inhibit radiolytic self-decomposition may be employed in accordance with this invention. Exemplary approaches to minimizing radiolytic self-decomposition that may be employed in accordance with this invention include, but are not limited to, reducing the molar specific activity of the compound, dispersing the compound in a solvent or solid dilutent, adding free-radical inhibitors, adding inhibitors against chemical decomposition, and storing the compound at low temperatures. In a preferred embodiment, a compound of the formula (VI): 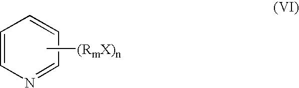
where R is C₁ to C₄ alkyene which may be OH substituted; m is 0 or 1; X is carboxyl or sulphonyl; and n is 1, 2, or 3; is added to the radiolabeled compound. The compound may be added to a solution containing the radiolabeled compound. Additionally, anti-oxidants, more particularly non-volatile anti-oxidants, may be included with the stabilizing compound. Examples of appropriate antioxidants include, but are not limited to, dithiothreitol and ascorbic acid.

In another preferred embodiment for stabilizing the radiolabeled complexing targeting moiety, a compound selected from the group consisting of (i) heteroaryls, (ii) aryls, and (iii) alkylamines is added to the solution containing the radiolabeled compound. The heteroaryls have at least one nitrogen atom and are substituted with at least one sulfur-containing moiety selected from thiol and thiocarbonyl, provided that the nitrogen atoms are not adjacent to one another. The aryls are substituted with at least one nitrogen-containing moiety selected from amino and isothiocyanate and with at least one sulfur-containing moiety selected sulfonamide, sulfonate, and thiol. The alkylamines have at least one to four carbon atoms and are substituted with at least one sulfur-containing moiety selected from thioacid and thiocarbonyl, provided that when the sulfur-containing moiety is a thioacid then the aminoalkyl contains only one nitrogen atom.

The radiopharmaceuticals produced by practice of the present invention may be used in diagnostic or therapeutic medical procedures. For example, the radiopharmaceutical may be used as an imaging contrast agent to produce PET or other radiographic images. Alternatively, the radiopharmaceutical may be used as a therapeutic agent that delivers doses of radiation to specific structures or sites of physiological activity in the body. One skilled in the art will appreciate other pharmacological uses of the radiopharmaceutical.

The invention now will be explained by reference to the following non-limiting examples.

### EXAMPLE 1

Peptide nucleic acids (PNA) may be synthesized using standard solid-phase synthesis techniques with Fmoc protecting groups on the terminal amino groups of the PNA monomers (commercially available as Expedite® Fmoc PNA Monomers from Applied Biosystems, Foster City, Calif.). 5-(4-Fmoc-aminoethyl-3,5-dimethoxyphenoxy)-valeric acid-MBHA resin (commercially available as PAL from Applied Biosystems, Foster City, Calif.) may be chosen as the polymer substrate. The side-chains of the PNA monomers may be protected using Bhoc groups. The PNAs may be synthesized using a solid-phase peptide synthesizer such as the Symphony® synthesizer (commercially available from Rainin Instrument Company, Woburn, Mass.). Prior to any chemistry, the resin may be swelled in dichloromethane (DCM) and subsequently exchanged out with N,N-dimethylformamide (DMF). The Fmoc-protected amine on the resin may be deprotected by washing with 20% piperidine in DMF. The resin then may be washed with DMF and DCM. After all subsequent reactions, the resin also may be thoroughly washed with DMF and DCM.

Each peptide coupling reaction may be carried out in N-methylpyrrolidinone (NMP) with excess equivalents of monomer dissolved in NMP. HATU (O-(7-Azabenzotriazol-1-yl)-N,N,N′,N′-tetramethyluronium hexafluorophosphate) may be used as the coupling reagent, with DIEA (N,N-diisopropylethylamine) in pyridine as the base. For each monomer coupling step, the coupling agent-DIEA solution may be delivered to the monomer solution and a reaction carried out outside the synthesizer while the resin is soaked in NMP. The coupling agent-activated monomer solution then may be added to the resin and the coupling reaction carried out.

Following each coupling reaction, the N-terminal Fmoc-protected amine may be deprotected by applying 20% piperidine.

To conjugate TETA with the terminal amino group of the resin-bound PNA, a premixed solution of TETA-t-Bu₃ dissolved in NMP, excess equivalents of HATU, and DIEA in pyridine may be added and the reaction carried out.

The resin, still on the peptide synthesizer, may be rinsed thoroughly with DMF and methylene chloride, dried under nitrogen, and lyophilized in preparation of resin cleavage. To cleave the PNAs from the resin, a cocktail consisting of TFA (trifluoroacetic acid) and 20% m-cresol may be used. The resin and cocktail may be stirred at room temperature for a period of time. The resin beads then may be filtered off using glass wool, followed by rinsing with TFA. The PNA may be precipitated with ice-cold ether and centrifuged until the precipitate forms at the bottom of the centrifuge tube. The pellet may be dried in the lyophilizer.

### EXAMPLE 2

A PNA may be produced as described in Example 1.

To conjugate TETA with the side chain of a lysine amino acid conjugated to the terminus of the resin-bound PNA, a premixed solution of N^{α}-Ac—N^{ε}-Fmoc-L-lysine (prepared in two steps from N^{α}-Boc-N^{ε}-Fmoc-L-lysine), HATU, and DIEA as in Example 1 may be added to the resin-bound PNA. The reaction may be carried out to form a PNA-lysine conjugate. The Fmoc group may be deprotected with 20% piperidine in DMF. After washing with DMF, a premixed solution of excess equivalents of TETA-t-Bu₃ and excess equivalents of HATU may be dissolved in N-methylmorpholine (NMM) and DMF and added to the resin-bound PNA-lysine conjugate.

Washing, rinsing, cleavage, and precipitation of the PNA may be completed as in Example 1.

### EXAMPLE 3

A PNA may be produced as described in Example 1, with the exception that an additional lysine monomeric unit may be introduced into the PNA chain during synthesis. Introduction of the lysine into the PNA chain during synthesis may be accomplished by using a N^{α}-Fmoc-N^{ε}-Mtt-L-lysine monomer during one of the synthesis steps.

To conjugate TETA with the side chain of the non-terminal lysine amino acid in the PNA, Mtt may be selectively deprotected with 3% TFA and 5% i—Pr₃SiH in DCM followed by extensive washing. The deprotection may be done either at the time of lysine coupling, or at any subsequent point of the synthesis of the resin-bound PNA. Following the deprotection, coupling of TETA-t-Bu₃ may be accomplished as in Example 2.

Washing, rinsing, cleavage, and precipitation of the PNA may be completed as in Example 1.

While the description of the present invention presented above has been described with reference to particularly preferred embodiments, it is recognized that similar advantages may be obtained by other embodiments. It will be evident to those skilled in the art that various changes and modifications can be made without departing from the spirit and scope of the present invention, and all such modifications are within the scope of this invention.

1. A method for conjugation of one or more complexing agents with a targeting moiety comprising:
attaching the targeting moiety to a substrate to form a targeting moiety—substrate component; and
conjugating the one or more complexing agents to the targeting moiety—substrate component at one or more free amino groups of the targeting moiety—substrate component to form a complexing targeting moiety—substrate component;
wherein the targeting moiety comprises one or more monomeric units.

2. The method of claim 1, wherein the targeting moiety is selected from the group consisting of natural amino acids, unnatural amino acids, peptides, peptide nucleic acids, nucleotides, and analogs and derivatives thereof.

3. The method of claim 1, wherein at least one of the one or more monomeric units of the targeting moiety is selected from the group consisting of lysine, lysine derivatives, and lysine analogs.

4. The method of claim 1, wherein the one or more free amino groups of the targeting moiety—substrate component are located at a terminus or a side chain of the targeting moiety—substrate component.

5. The method of claim 1, further comprising cleaving the complexing targeting moiety from the substrate.

6. The method of claim 1, where the targeting moiety is a compound of formula (V): 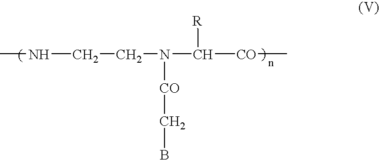
where B is a heterocyclic base independently selected from the group consisting of adenine, guanine, cytosine, thymine, and uracil;
R is independently selected from the group consisting of hydrogen and the side groups covalently bonded to α-carbons of the naturally occurring α-amino acids; and
n is an integer in a range of from about 4 to about 20, inclusive.

7. The method of claim 1, further comprising linking one or more additional monomeric units to the targeting moiety—substrate component before conjugating the one or more complexing agents to the targeting moiety—substrate component.

8. The method of claim 7, wherein the one or more additional monomeric units prior to linking to the targeting moiety—substrate component each has only one free amino-reactive group.

9. The method of claim 7, wherein the one or more additional monomeric units are independently selected from the group consisting of natural amino acids, unnatural amino acids, peptides, peptide nucleic acids, nucleotides, and analogs and derivatives thereof.

10. The method of claim 7, wherein at least one of the one or more additional monomeric units is selected from the group consisting of lysine, lysine derivatives, and lysine analogs.

11. The method of claim 1, further comprising linking one or more additional monomeric units to the complexing targeting moiety—substrate component.

12. The method of claim 10, wherein the one or more additional monomeric units prior to linking to the complexing targeting moiety—substrate component each has only one free amino-reactive group.

13. The method of claim 10, wherein the one or more additional monomeric units are independently selected from the group consisting of natural amino acids, unnatural amino acids, peptides, peptide nucleic acids, nucleotides, and analogs and derivatives thereof.

14. The method of claim 10, wherein at least one of the one or more additional monomeric units is selected from the group consisting of lysine, lysine derivatives, and lysine analogs.

15. The method of claim 1, wherein the complexing agent is a compound of formula (III) 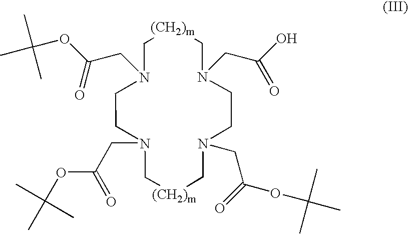
where m is 1 or 2.

16. The method of claim 1, wherein the one or more complexing agents are independently selected from the group of compounds consisting of diethylenetriamine-pentaacetic acid (“DTPA”); 1,4,7,10-tetraazacyclododecane-N,N′,N″,N′″-tetraacetic acid (“DOTA”); p-isothiocyanatobenzyl-1,4,7,10-tetraazacyclododecan-1,4,7,10-tetraacetic acid (“pSCN-Bz-DOTA”); 1,4,7,10-tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (“NOTA”); 1,4,8,11-tetraazacyclotetradecane-N,N′,N″,N′″-tetraacetic acid (“TETA”); triethylene tetraamine hexaacetic acid (“TTHA”); trans-1,2-diaminohexane tetraacetic acid (“CYDTA”); 1,4,7,10-tetraazacyclododecane-1-(2-hydroxypropyl)4,7,10-triacetic acid (“HP-DO3A”);
trans-cyclohexane-diamine tetraacetic acid (“CDTA”); trans(1,2)-cyclohexane diethylene triamine pentaacetic acid (“CDTPA”); 1-oxa-4,7,10-triazacyclododecane-N,N′,N″-triacetic acid (“OTTA”); 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives, analogs, and mixtures thereof.

17. The method of claim 1, wherein the complexing agent binds copper-64.

18. The method of claim 1, wherein the complexing agent binds a radioactive metallic ion selected from the group consisting of: actinium-225, bismuth-212, arsenic-72, indium-110, indium-111, indium-113m, gallium-67, gallium-68, strontium-83, zirconium-89, ruthenium-95, ruthenium-97, ruthenium-103, ruthenium-105, mercury-107, mercury-203, rhenium-186, rhenium-1881 tellurium-121 m, tellurium-122m, tellurium-125m, thulium-165, thulium-167, thulium-168, technetium-94m, technetium-99m, silver-111, platinum-197, palladium-109, copper-62, copper-64, copper-67, yttrium-86, yttrium-90, scandium-47, samarium-153, lutetium-177) rhodium-105, praseodymium-142, praseodymium-143, terbium-161, holmium-166, gold-199, cobalt-57, cobalt-58, chromium-51, iron-59, selenium-75, thallium-201, and ytterbium-169.
